(19) United States
(12) Patent Application Publication
(10) Pub. No.: US 2005/0058698 A1
(43) Pub. Date: Mar. 17, 2005

(54) Pharmaceutically acceptable phosphate-glycerol carrying bodies and uses relating to Parkinson's Disease

(76) Inventors: Yvonne Mairead Nolan, Cork (IE); Anthony E. Bolton, Bakewell (GB); Arkady Mandel, North York (CA)

Correspondence Address: FOLEY & LARDNER LLP;THREE PALO ALTO SQUARE, 3000 EL CAMINO REAL, SUITE 100, PALO ALTO, CA, 94306, US

(21) Appl. No.: 10/894,649

(22) Filed: Jul. 19, 2004

Related U.S. Application Data
(63) Continuation-in-part of application No. 10/348,601, filed on Jan. 21, 2003.
(60) Provisional application No. 60/421,116, filed on Jan. 22, 2002.
(60) Provisional application No. 60/351,427, filed on Jan. 28, 2002.
(60) Provisional application No. 60/364,620, filed on Mar. 18, 2002.
(60) Provisional application No. 60/372,106, filed on Apr. 15, 2002.
(60) Provisional application No. 60/400,857, filed on Aug. 2, 2002.

(30) Foreign Application Priority Data
Jan. 21, 2002 (CA) 2,368,656

(51) Int. Cl. A61K 9/127; C12N 5/00; C12N 15/88

(52) U.S. Cl. 424/450; 435/458

(57) ABSTRACT

This invention relates to three-dimensional synthetic and semi-synthetic compositions having biological activity, and to the uses thereof in the treatment and/or prophylaxis of various disorders in mammalian patients. More particularly it relates to preparations and uses of synthetic and semi-synthetic bodies, such as liposomes, which after introduction into the body of a patient, produce beneficial anti-inflammatory, organ protective and immune regulatory effects. The invention also relates to treatments and compositions for alleviating inflammatory and autoimmune diseases and their symptoms.

### CROSS-REFERENCE TO RELATED APPLICATIONS

This application is a continuation-in-part application of U.S. patent application Ser. No. 10/348,601, filed Jan. 21, 2003, which claims priority under 35 U.S.C. §119(a) to Canadian Application No. 2,368,656, filed on Jan. 21, 2002, and which further claims priority under 35 U.S.C. § 119(e) to the following applications: U.S. Provisional Application No. 60/421,116, which was converted pursuant to 37 C.F.R. § 1.53(c)(2)(i) from U.S. patent application Ser. No. 10/051,381, filed Jan. 22, 2002; U.S. Provisional Application No. 60/351,427, filed Jan. 28, 2002; U.S. Provisional Application No. 60/364,620, filed Mar. 18, 2002; U.S. Provisional Application 60/372,106, filed Apr. 15, 2002 and U.S. Provisional Application No. 60/400,857, filed Aug. 2, 2002. The foregoing applications are herein incorporated by reference in their entireties.

### FIELD OF THE INVENTION

This invention relates to three-dimensional synthetic and semi-synthetic compositions having biological activity, and to the uses thereof in the treatment and/or prophylaxis of various disorders in mammalian patients. More particularly it relates to preparations and uses of synthetic and semi-synthetic bodies, which after introduction into the body of a patient, produce beneficial anti-inflammatory, organ protective and immune regulatory effects. The invention also relates to treatments and compositions for alleviating inflammatory and autoimmune diseases and their symptoms, including the inflammatory component of brain disorders.

### REFERENCES

All of the publications, patents and patent applications listed below are herein incorporated by reference in their entirety to the same extent as is if each individual publication, patent or patent application was specifically and individually indicated to be incorporated by reference in its entirety.
1. U.S. Pat. No. 4,485,054, issued Nov. 27, 1984, to Mezei, et al.
2. U.S. Pat. No. 4,496,787, issued Jan. 29, 1985, to Touchais, et al.
3. U.S. Pat. No. 4,812,314, issued Mar. 14, 1989 to Barenholz,
4. U.S. Pat. No. 4,938,763, issued Jul. 3, 1990 to Dunn, et al.
5. U.S. Pat. No. 4,946,787, issued Aug. 7, 1990, to Eppstein, et al.
6. U.S. Pat. No. 5,188,951, issued Feb. 23, 1993, to Tremblay, et al.
7. U.S. Pat. No. 5,252,263, issued Oct. 12, 1993, to Hope, et al.
8. U.S. Pat. No. 5,376,452, issued Dec. 27, 1994, to Hope, et al.
9. U.S. Pat. No. 5,736,157, issued Apr. 7, 1998, to Williams.
10. U.S. Pat. No. 5,741,514, issued Apr. 21, 1998, to Barenholz, et al.
11. U.S. Pat. No. 5,746,223, issued May 5, 1998, to Williams.
12. U.S. Pat. No. 5,843,474, issued Dec. 1, 1998, to Williams.
13. U.S. Pat. No. 5,858,400, issued Jan. 12, 1999, to Williams.
14. U.S. Pat. No. 6,297,870, issued Oct. 2, 2001, to Nanba.
15. U.S. Pat. No. 6,312,719, issued Nov. 6, 2001, to Hope, et al.
16. International Publication No. WO 01/66785, published Sep. 13, 2001.
17. International Patent Application PCT/CA02/01398 to Vasogen Ireland Limited.
18. Barenholz et al. “Liposomes as Pharmaceutical Dosage Forms”
19. New, R. C. “Liposomes: A Practical Approach”, IRL Press at Oxford University Press (1990).
20. Richard Harrigan—1992 University of British Columbia PhD Thesis “Transmembrane pH gradients in liposomes (microform): drug-vesicle interactions and proton flux”, published by National Library of Canada, (1993); University Microfilms order no. UMI00406756; Canadian no. 942042220, ISBN 0315796936.
21. Griffin WST et al. “Brain interleukin 1 and S-100 immunoreactivity are elevated in Down Syndrome and Alzheimer Disease.” Proceedings of the National Academy of Sciences USA. 86: 7611-7615 (1989).
22. Bliss, T. V. P., et al. “A synaptic model of memory: long-term potentiation in the hippocampus.” Nature. 361: 31-39 (1993).
23. Murray, C. A., et al. “Evidence that increase hippocampal expression of the cytokine interleukin-1B is a common trigger for age and stress-induced impairments in long-term potentiation.” J. Neuroscience. 18: 2974-2981 (1998).
24. Mogi, M., et al. “Interleukin (IL)-1 beta, IL-1, IL-4, IL-6 and transforming growth factor-alpha levels are elevated in ventricular cerebrospinal fluid in juvenile parkinsonism and Parkinson's Disease.” Neuroscience Letters. 211: 13-16 (1996).
25. Giannessi D, Del Ry S, Vitale R L “The role of endothelins and their receptors in heart failure.” Pharmacol Res 2001 February 43:2 111-26.
26. Van de Stolpe A, Van der Saag P T, “Intercellular adhesion molecule-1” J. Mol. Med. (1996) 74:112-33.

### BACKGROUND OF THE INVENTION

Professional antigen-presenting cells (APCs), including dendritic cells (DCs) and macrophages (Mph), actively capture and process antigens (Ags), clear cell debris, and remove infectious organisms and dying cells, including the residues from dying cells. During this process, APCs can stimulate the production of either inflammatory Th1 pro-inflammatory cytokines (IL-12, IL-1, INF-γ, TNF-α, etc.); or regulatory Th2/Th3 cytokines (such as IL-10, TGF-β, IL-4, etc.) dominated responses; depending on the nature of the antigen (Ag) or phagocytosed material and the level of APC maturation/activation.

APCs remove cellular debris, some of which is derived from cell membranes of the body, some from bacterial and parasitic infections and commensal organisms, such as gut bacteria. While some of this cellular debris will initiate a pro-inflammatory response, some initiates a protective and anti-inflammatory response.

Producing an anti-inflammatory effect is often desirable when treating a number of disorders. This can be accomplished by inhibiting pro-inflammatory cytokines, such as TNF-α, or promoting anti-inflammatory cytokines. Various anti-inflammatory agents are known in the art, such as agents that reduce leukocyte extravasation, corticosteroids and nonsteroidal anti-inflammatory drugs. However, there is a need to provide more effective anti-inflammatory agents.

### SUMMARY OF THE INVENTION

This invention is directed to the discovery that pharmaceutically acceptable bodies, such as liposomes, beads or similar particles, which comprise phosphate-glycerol groups, will, upon administration to a mammalian patient, cause an anti-inflammatory effect and therefore may be used to treat a number of diseases. These bodies may further comprise as a minor component an inactive constituent, and/or constituent which is active through a different mechanism.

In a preferred embodiment, the invention is directed to a composition of matter capable of producing an anti-inflammatory response in vivo in a mammal, said composition comprising pharmaceutically acceptable bodies of a size from about 20 nanometers (nm) to 500 micrometers (μm), comprising a plurality of phosphate-glycerol groups or groups convertible to such groups. Preferably, the bodies are essentially free of non-lipid pharmaceutically active entities. Preferably the phosphate-glycerol groups constitute 60%-100% of the active groups on the bodies. Following administration to a mammal, the bodies, through the phosphate-glycerol groups, are believed to interact with the immune system. As a result, when so administered an anti-inflammatory response is elicited.

In another embodiment, this invention is directed to a three-dimensional synthetic or semi-synthetic body, otherwise referred to herein as pharmaceutically acceptable bodies, having a size ranging from 20 nm to 500 μm, and having been modified to comprise, as a major component, at least one anti-inflammatory promoting ligand wherein said ligand has phosphate-glycerol groups.

In still another embodiment, this invention is directed to three-dimensional synthetic and semi-synthetic bodies, otherwise referred to herein as pharmaceutically acceptable bodies, having sizes ranging from 20 nm to 500 μm, and having phosphate-glycerol groups on the surface thereof.

In another preferred embodiment, the invention is directed to methods for the prophylaxis, treatment or delay in the progression of Parkinson's disease in a mammalian patient which comprises reducing the level of TNF-α in the cortex of the mammalian patient by administering to the patient a composition of the invention.

Optionally, the bodies described above may additionally comprise an inactive constituent surface group and/or a constituent surface group, which is active through another mechanism, e.g. phosphatidylserine. (See, e.g. Fadok et al., International Publication WO 01/66785).

In another embodiment, this invention is directed to lyophilized or freeze-dried pharmaceutically acceptable bodies carrying phosphate-glycerol groups or groups convertible to phosphate-glycerol groups, and kits comprising lyophilized or freeze dried bodies comprising phosphate-glycerol groups, or groups convertible to phosphate-glycerol groups, and a pharmaceutically acceptable carrier.

### BRIEF DESCRIPTION OF THE DRAWINGS

FIG. 1 is a bar graph presentation of the results of Example 1 below, murine contact hypersensitivity (CHS, acute T-cell mediated inflammatory model) experiments using liposomes in accordance with a preferred embodiment of the invention, in comparison with other liposomes and controls.

FIG. 2 is a similar graphical presentation, showing the use of liposomes of various phosphatidylglycerol (PG) contents, in the murine CHS model, Example 2 below.

FIG. 3 is a similar graphical presentation of the results of Example 3 below where different concentrations of 75% PG liposomes were used in the murine CHS model.

FIG. 4 is a similar graphical presentation of the results of Example 4 below, where different concentrations of 100% PG liposomes were used in the murine CHS model.

FIG. 5 is a similar graphical presentation of the results of Example 5 below, using liposomes of different sizes in the CHS model.

FIG. 6 is a similar graphical presentation of the results of Example 6 below, using a murine model of delayed type hypersensitivity (DHS, chronic T-cell mediated inflammatory model).

FIG. 7 is a similar graphical presentation of the results of Example 7 below, cardiolipin liposomes in a DHS murine model.

FIG. 8 is a similar graphical presentation of the results of Example 8 below, cardiolipin liposomes in a CHS murine model.

FIG. 9 shows the change in the percentage of excitatory post-synaptic potential (EPSP) slope in control and treated mice, which is indicative of the effect on long term potentiation (LTP), Example 9.

FIG. 10 displays the data shown in FIG. 9 in the format of a bar chart, Example 9 below.

FIG. 11 Shows the difference in the concentration of the anti-inflammatory cytokine IL-4 in the hippocampus of control and treated animals, Example 10 below.

FIG. 12 shows the difference in the concentration of the pro-inflammatory cytokine IL-1β in a single cell suspension of spleen cells of control and treated animals, Example 11 below.

FIG. 13 shows the difference in the concentration of TNF-α in the U937 monocyte cell line treated with varying concentration of 75% PG liposomes, Example 12 below.

FIG. 14 is a graphical presentation of the results of Example 13 below, endothelin-1 content in ears of mice treated according to a preferred embodiment of the invention versus control.

FIG. 15 is a graphical presentation of the results of Example 14, ICAM-1 positive cells from HUVEC cultures in the presence and absence of compositions of the preferred embodiment of the invention.

FIG. 16 is a graphical presentation of the behavioral results of animals treated as described in Example 16, a model of Parkinson's Disease.

FIGS. 17 and 18 are graphical presentation of the results of analysis of cortical tissue for TNF-α content from animals treated as described in Example 1, the model of Parkinson's Disease.

### DESCRIPTION OF PREFERRED EMBODIMENTS

According to the present invention, pharmaceutically acceptable bodies carrying phosphate-glycerol groups on their surface are administered to patients. Without being limited to any theory, is believed that these bodies interact with the immune system of the patient with accompanying beneficial effects such as inhibition of pro-inflammatory cytokines in vivo and/or promotion of anti-inflammatory cytokines. The reacting cells may be immune cells such as professional or non-professional antigen presenting cells, endothelial cells, regulatory cells such as NK-T cells and others.

1. Definitions

This section sets forth certain defined terms; other terms used herein are defined in context and/or have the meanings generally attributable to them in standard usage by those skilled in the art.

The term “biocompatible” refers to substances that, in the amount employed, are either non-toxic or have acceptable toxicity profiles such that their use in vivo is acceptable.

The terms “liposomes” and “lipid vesicles” refer to sealed membrane sacs, having diameters in the micron or sub-micron range, the walls of which consist of layers, typically bilayers, of suitable, membrane-forming amphiphiles. They normally contain an aqueous medium.

The term “pharmaceutically acceptable” has a meaning that is similar to the meaning of the term “biocompatible.” As used in relation to “pharmaceutically acceptable bodies” herein, it refers to bodies of the invention comprised of one or more materials which are suitable for administration to a mammal, preferably a human, in vivo, according to the method of administration specified (e.g., intramuscular, intravenous, subcutaneous, topical, oral, and the like).

The term “phosphate choline” refers to the group —O—P(═O)(OH)—O—CH₂—CH₂—N⁺—(CH₃)₃, which can attached to lipids to form “phosphatidylcholine” (PC) as shown in the following structure: 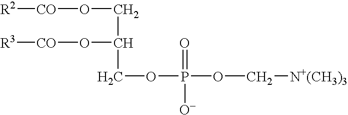
and salts thereof, wherein R² and R³ are independently selected from C₁-C₂₄ hydrocarbon chains, saturated or unsaturated, straight chain or containing a limited amount of branching wherein at least one chain has from 10-24 carbon atoms.

The term “phosphate-glycerol-carrying bodies” refers to biocompatible, pharmaceutically-acceptable, three-dimensional bodies having on their surfaces phosphate-glycerol groups or groups that can be converted to phosphate-glycerol groups, as described herein.

A “phosphate-glycerol group” is a group having the general structure: O—P(═O)(OH)—O—CH₂CH(OH)CH₂OH, and derivatives thereof, including, but not limited to groups in which the negatively charged oxygen of the phosphate group of the phosphate-glycerol group is converted to a phosphate ester group (e.g., L-OP(O)(OR′)(OR″), where L is the remainder of the phosphate-glycerol group, R′ is —CH₂CH(OH)CH₂OH and R″ is alkyl of from 1 to 4 carbon atoms, or a hydroxyl substituted alkyl of from 2 to 4 carbon atoms, and 1 to 3 hydroxyl groups provided that R″ is more readily hydrolyzed in vivo than the R′ group; to a diphosphate group including diphosphate esters (e.g., L-OP(O)(OR′)OP(O)(OR″)₂ wherein L and R′ are as defined above and each R″ is independently hydrogen, alkyl of from 1 to 4 carbon atoms, or a hydroxyl substituted alkyl of from 2 to 4 carbon atoms and 1 to 3 hydroxyl groups, provided that the second phosphate [—P(O)(OR″)₂] is more readily hydrolyzed in vivo than the R′ group; or to a triphosphate group including triphosphate esters (e.g., L-OP(O)(OR′)OP(O)(OR″)OP(O)(OR″)₂ wherein L and R′ are defined as above and each R″ is independently hydrogen, alkyl of from 1 to 4 carbon atoms, or a hydroxyl substituted alkyl of from 2 to 4 carbon atoms and 1 to 3 hydroxyl groups provided that the second and third phosphate groups are more readily hydrolyzed in vivo than the R′ group; and the like. Such synthetically altered phosphate-glycerol groups are capable of expressing phosphate-glycerol in vivo and, accordingly, such altered groups are phosphate-glycerol convertible groups within the scope of the invention. A specific example of a phosphate-glycerol group is the compound phosphatidylglycerol (PG), further defined herein.

“Phosphatidylglycerol” is also abbreviated herein as “PG.” This term is intended to cover phospholipids carrying a phosphate-glycerol group with a wide range of at least one fatty acid chain provided that the resulting PG entity can participate as a structural component of a liposome. Chemically, PG has a phosphate-glycerol group and a pair of similar, but different fatty acid side chains. Preferably, such PG compounds can be represented by the Formula I: 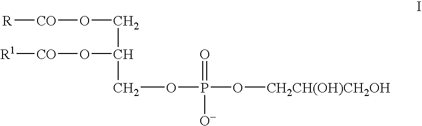
where R and R¹ are independently selected from C₁-C₂₄ hydrocarbon chains, saturated or unsaturated, straight chain or containing a limited amount of branching wherein at least one chain has from 10 to 24 carbon atoms. R and R¹ can be varied to include two or one lipid chain(s), which can be the same or different, provided they fulfill the structural function. As mentioned above, the fatty acid side chains may be from about 10 to about 24 carbon atoms in length, saturated, mono-unsaturated or polyunsaturated, straight-chain or with a limited amount of branching. Laurate (C12), myristate (C14), palmitate (C16), stearate (C18), arachidate (C20), behenate (C22) and lignocerate (C24) are examples of useful saturated fatty acid side chains for the PG for use in the present invention. Palmitoleate (C15), oleate (C18) are examples of suitable mono-unsaturated fatty acid side chains. Linoleate (C18), linolenate (C18) and arachidonate (C20) are examples of suitable poly-unsaturated fatty acid side chains for use in PG in the compositions of the present invention. Phospholipids with a single such fatty acid side chain, also useful in the present invention, are known as lysophospholipids.

The term PG also includes dimeric forms of PG, namely cardiolipin, but other dimers of Formula I are also suitable. Preferably, such dimers are not synthetically cross-linked with a synthetic cross-linking agent, such as maleimide but rather are cross-linked by removal of a glycerol unit as described by Lehninger, Biochemistry and depicted in the reaction below:

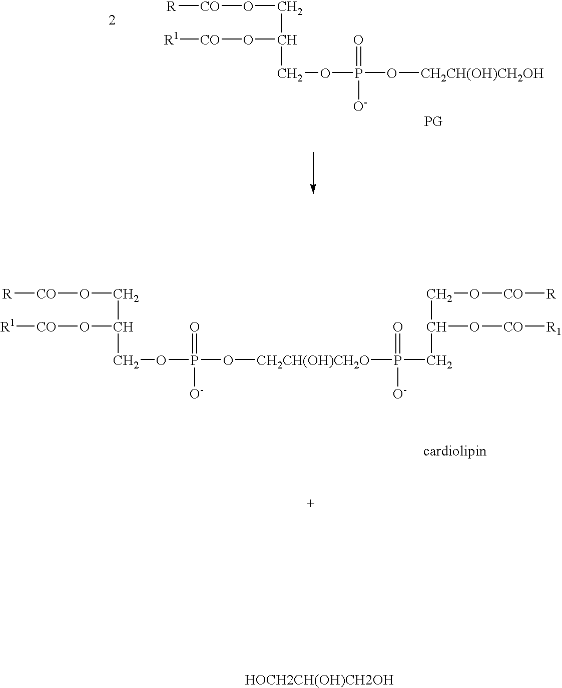

Purified forms of phosphatidylglycerol are commercially available, for example, from Sigma-Aldrich (St. Louis, Mo.). Alternatively, PG can be produced, for example, by treating the naturally occurring dimeric form of phosphatidylglycerol, cardiolipin, with phospholipase D. It can also be prepared by enzymatic synthesis from phosphatidyl choline using phospholipase D. See, for example, U.S. Pat. No. 5,188,951 (Tremblay et al.), incorporated herein by reference.

“PG-carrying bodies” are three-dimensional bodies, as described above, that have surface PG molecules. By way of example, PG can form the membrane of a liposome, either as the sole constituent of the membrane or as a major or minor component thereof, with other phospholipids and/or membrane forming materials.

The term “phosphatidylserine” or “PS” is intended to cover phosphatidyl serine and analogs/derivatives thereof.

In the context of the present invention, “three-dimensional bodies” refer to biocompatible synthetic or semi-synthetic entities, including but not limited to liposomes, solid beads, hollow beads, filled beads, particles, granules and microspheres of biocompatible materials, natural or synthetic, as commonly used in the pharmaceutical industry. Liposomes may be formed of lipids, including phosphatidylglycerol (PG). Beads may be solid or hollow, or filled with a biocompatible material. Such bodies have shapes that are typically, but not exclusively spheroidal, cylindrical, ellipsoidal, including oblate and prolate spheroidal, serpentine, reniform and the like, and have sizes ranging from 200 nm to 500 μm, preferably measured along the longest axis.

“Treatment” includes, for example, a reduction in the number of symptoms, a decrease in the severity of at least one symptom of the particular disease or a delay in the further progression of at least one symptom of the particular disease.

2. Phosphate-Glycerol-Carrying Bodies

This section describes various embodiments of phosphate-glycerol-carrying bodies contemplated by the present invention, including specific embodiments thereof. With the guidance provided herein, persons having requisite skill in the art will readily understand how to make and use phosphate-glycerol-carrying bodies in accordance with the present invention.

In the context of the present invention, phosphate-glycerol-carrying bodies refer to biocompatible, pharmaceutically-acceptable, three-dimensional bodies having on their surfaces phosphate-glycerol groups or groups that can be converted to phosphate-glycerol groups, as described herein.
a. Phosphate-Glycerol Groups

According to a general feature of the invention, phosphate-glycerol groups useful in the present invention have the general structure:
O—P(═O)(OH)—O—CH₂CH(OH)CH₂OH

Such phosphate-glycerol groups include synthetically altered versions of the phosphate-glycerol group shown above, and may include all, part of or a modified version of the original phosphate-glycerol group.

Preferably the fatty acid side chains of the chosen PG will be suitable for formation of liposomes, and incorporation into the lipid membrane(s) forming such liposomes, as described in more detail below.

More generally, without being limited to any particular theory, it is believed that phosphate-glycerol groups according to the present invention are capable of interacting with one or more receptors present in relevant brain tissue, such as the hippocampus. A specific example of a phosphate-glycerol group is the compound phosphatidylglycerol (PG), described above.

PG groups of the present invention, including dimers thereof, are believed to act as ligands, binding to specific sites on a protein or other molecule (“PG receptor”) and, accordingly, PG (or derivatives or dimeric forms thereof) are sometimes referred to herein as a “ligand” or a “binding group.” Such binding is believed to take place through the phosphate-glycerol group —O—P(═O)(OH)—O—CH₂CH(OH)CH₂OH, which is sometimes referred to herein as the “head group,” “active group,” or “binding group,” while the fatty acid side chain(s) are believed to stabilize the group and/or, in the case of liposomal preparations, form the outer lipid layer or bilayer of the liposome. More generally, again without being limited to any particular theory, it is believed that phosphate-glycerol groups, including PG are capable of interacting with one or more receptors in the brain and that such interactions may provide positive effects on synaptic transmission, and, by extension, memory, as described herein.

As noted above, analogues of phosphatidylglycerol with modified active groups, which also interact with PG receptors on the antigen presenting cells, through the same receptor pathway as PG or otherwise resulting in an anti-inflammatory reaction in the recipient body are contemplated within the scope of the term phosphatidylglycerol. This includes, without limitation, compounds in which one or more of the hydroxyl groups and/or the phosphate group is derivatized, or in the form of a salt. Many such compounds form free hydroxyl groups in vivo, upon or subsequent to administration and, accordingly, comprise convertible PG groups.

b. Formation of Phosphate-Glycerol Carrying Bodies

Phosphate-glycerol carrying bodies are three-dimensional bodies that have surface phosphate-glycerol molecules. This section will describe general and exemplary phosphate-glycerol carrying bodies suitable for use in the present invention.

Generally, phosphate-glycerol carrying bodies of the present invention carry phosphate-glycerol molecules on their exterior surfaces to facilitate in vivo interaction of the binding groups.

Three-dimensional bodies are preferably formed to be of a size or sizes suitable for administration to a living subject, preferably by injection; hence such bodies will preferably be in the range of 20 to 1000 nm (0.02-1 micron), more preferably 20 to 500 nm (0.02-0.5 micron), and still more preferably 20-200 nm in diameter, where the diameter of the body is determined on its longest axis, in the case of non-spherical bodies. Suitable sizes are generally in accordance with blood cell sizes. While bodies of the invention have shapes that are typically, but not exclusively spheroidal, they can alternatively be cylindrical, ellipsoidal, including oblate and prolate spheroidal, serpentine, reniform in shape, or the like.

Suitable forms of bodies for use in the compositions of the present invention include, without limitation, particles, granules, microspheres or beads of biocompatible materials, natural or synthetic, such as polyethylene glycol, polyvinylpyrrolidone, polystyrene, and the like; polysaccharides such as hydroxethyl starch, hydroxyethylcellulose, agarose and the like; as are commonly used in the pharmaceutical industry. Preferably, such materials will have side-chains or moieties suitable for derivatization, so that a phosphate-glycerol group, such as PG, may be attached thereto, preferably by covalent bonding. Bodies of the invention may be solid or hollow, or filled with biocompatible material. They are modified as required so that they carry phosphate-glycerol molecules, such as PG on their surfaces. Methods for attaching phosphate-glycerol in general, and PG in particular, to a variety of substrates are known in the art.

In addition to the various bodies listed above, the liposome is a particularly useful form of body for use in the present invention. Liposomes are microscopic vesicles composed of amphiphilic molecules forming a monolayer or bilayer surrounding a central chamber, which may be fluid-filled. Amphophlilic molecules (also referred to as “amphiphiles”), are molecules that have a polar water-soluble group attached to a water-insoluble (lipophilic) hydrocarbon chain, such that a matrix of such molecules will typically form defined polar and apolar regions. Amphiphiles include naturally occurring lipids such as PG, phosphatidylserine, phosphatidylethanolamine, phosphatidylinositol, phosphatidylcholine, cholesterol, cardiolipin, ceramides and sphingomyelin, used alone or in admixture with one another. They can also be synthetic compounds such as polyoxyethylene alkyl ethers, polyoxyethylene alkyl esters and saccharosediesters.

Preferably, for use in forming liposomes, the amphiphilic molecules will include one or more forms of phospholipids of different head groups (e.g., phosphatidylglycerol, phosphatidylserine, phosphatidylcholine) and having a variety of fatty acid side chains, as described above, as well as other lipophilic molecules, such as cholesterol, sphingolipids and sterols.

In accordance with the present invention, phosphatidylglycerol (PG) will constitute the major portion or the entire portion of the liposome layer(s) or wall(s), oriented so that the phosphate-glycerol group portion thereof is presented exteriorly, as described above, while the fatty acid side chains form the structural wall. When, as in the present invention, the bilayer includes phospholipids, the resulting membrane is usually referred to as a “phospholipid bilayer,” regardless of the presence of non-phospholipid components therein.

Liposomes of the invention are typically formed from phospholipid bilayers or a plurality of concentric phospholipid bilayers which enclose aqueous phases. In some cases, the walls of the liposomes may be single layered; however, such liposomes (termed “single unilamellar vesicles” or “SUVs”) are generally much smaller (diameters less than about 70 nm) than those formed of bilayers, as described below. Liposomes formed in accordance with the present invention are designed to be biocompatible, biodegradable and non-toxic. Liposomes of this type are used in a number of pharmaceutical preparations currently on the market, typically carrying active drug molecules in their aqueous inner core regions. In the present invention, however, the liposomes are not filled with pharmaceutical preparation. The liposomes are active themselves, not acting as drug carrier.

Preferred PG-carrying liposomes of the present invention are constituted to the extent of 50%-100% by weight of phosphatidyl glycerol, the balance being phosphatidylcholine (PC) or other such biologically acceptable phospholipid(s). More preferred are liposomes constituted by PG to the extent of 65%-90% by weight, most preferably 70%-80% by weight, with the single most preferred embodiment, on the basis of current experimental experience, being PG 75% by weight, the balance being other phospholipids such as PC. Such liposomes are prepared from mixtures of the appropriate amounts of phospholipids as starting materials, by known methods. According to an important feature of the invention, PG-carrying bodies comprise less than 50%, preferably less than 40%, still preferably less than 25% and even still preferably less than 10% phosphatidyl serine.

The present invention contemplates the use, as PG-carrying bodies, not only of those liposomes having PG as a membrane constituent, but also liposomes having non-PG membrane substituents that carry on their external surface molecules of phosphate-glycerol, either as monomers or oligomers (as distinguished from phosphatidylglycerol), e.g., chemically attached by chemical modification of the liposome surface of the body, such as the surface of the liposome, making the phosphate-glycerol groups available for subsequent interaction. Because of the inclusion of phosphate-glycerol on the surface of such molecules, they are included within the definition of PG-carrying bodies.

Liposomes may be prepared by a variety of techniques known in the art, such as those detailed in Szoka et al. (Ann. Rev. Biophys. Bioeng. 9:467 (1980)). Depending on the method used for forming the liposomes, as well as any after-formation processing, liposomes may be formed in a variety of sizes and configurations. Methods of preparing liposomes of the appropriate size are known in the art and do not form part of this invention. Reference may be made to various textbooks and literature articles on the subject, for example, the review article by Yechezkel Barenholz and Daan J. A. Chromeline, and literature cited therein, for example New, R. C. (1990), and Nassander, U. K., et al. (1990), and Barenholz, Y and Lichtenberg, D., Liposomes: preparation, characterization, and preservation. Methods Biochem Anal. (1988) 33:337-462.

Multilamellar vesicles (MLV's) can be formed by simple lipid-film hydration techniques according to methods known in the art. In this procedure, a mixture of liposome-forming lipids is dissolved in a suitable organic solvent. The mixture is evaporated in a vessel to form a thin film on the inner surface of the vessel, to which an aqueous medium is then added. The lipid film hydrates to form MLV's, typically with sizes between about 100-1000 nm (0.1 to 10 microns) in diameter.

A related, reverse evaporation phase (REV) technique can also be used to form unilamellar liposomes in the micron diameter size range. The REV technique involves dissolving the selected lipid components, in an organic solvent, such as diethyl ether, in a glass boiling tube and rapidly injecting an aqueous solution, into the tube, through a small gauge passage, such as a 23-gauge hypodermic needle. The tube is then sealed and sonicated in a bath sonicator. The contents of the tube are alternately evaporated under vacuum and vigorously mixed, to form a final liposomal suspension.

By way of example, but not limitation, Example 1 provides a detailed description of a method of preparing a PG-liposomal preparation for use in the present invention.

The diameters of the PG-carrying liposomes of the preferred embodiment of this invention range from about 20 nm to about 1000 nm, more preferably from about 20 nm to about 500 nm, and most preferably from about 20 nm to about 200 nm. Such preferred diameters will correspond to the diameters of mammalian apoptotic bodies, such as may be apprised from the art.

One effective sizing method for REV's and MLV's involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the largest sizes of liposomes produced by extrusion through that membrane, particularly where the preparation is extruded two or more times through the same membrane. This method of liposome sizing is used in preparing homogeneous-size REV and MLV compositions. U.S. Pat. Nos. 4,737,323 and 4,927,637, incorporated herein by reference, describe methods for producing a suspension of liposomes having uniform sizes in the range of 0.1-0.4 μm (100-400 nm) using as a starting material liposomes having diameters in the range of 1 μm. Homogenization methods are also useful for down-sizing liposomes to sizes of 100 nm or less (Martin, F. J. (1990) In: Specialized Drug Delivery Systems—Manufacturing and Production Technology, P. Tyle (ed.) Marcel Dekker, New York, pp. 267-316.). Another way to reduce liposomal size is by application of high pressures to the liposomal preparation, as in a French Press.

Liposomes can be prepared to have substantially homogeneous sizes of single, bi-layer vesicles in a selected size range between about 0.07 and 0.2 microns (70-200 nm) in diameter, according to methods known in the art. In particular, liposomes in this size range are readily able to extravasate through blood vessel epithelial cells into surrounding tissues. A further advantage is that they can be sterilized by simple filtration methods known in the art.

Whilst a preferred embodiment of PG-carrying bodies for use in the present invention is liposomes with PG presented on the external surface thereof, it is understood that the PG-carrying body is not limited to a liposomal structure, as mentioned above.

3. Dosages and Modes of Administration

The phosphate-glycerol-carrying bodies of the invention may be administered to the patient by any suitable route of administration, including oral, nasal, topical, rectal, intravenous, subcutaneous and intramuscularly. At present, intramuscular administration is preferred, especially in conjunction with PG-liposomes.

It is contemplated that the patient may be a mammal, including but not limited to humans and domestic animals such as cows, horses, pigs, dogs, cats and the like.

The PG-carrying bodies may be suspended in a pharmaceutically acceptable carrier, such as physiological sterile saline, sterile water, pyrogen-free water, isotonic saline, and phosphate buffer solutions, as well as other non-toxic compatible substances used in pharmaceutical formulations. Preferably, PG-carrying bodies are constituted into a liquid suspension in a biocompatible liquid such as physiological saline and administered to the patient in any appropriate route which introduces it to the immune system, such as intra-arterially, intravenously, intra-arterially or most preferably intramuscularly or subcutaneously.

The quantities of PG-carrying bodies to be administered will vary depending on the identity and characteristics of the patient. It is important that the effective amount of PG-bodies is non-toxic to the patient. The most effective amounts are unexpectedly small. When using intra-arterial, intravenous, subcutaneous or intramuscular administration of a liquid suspension of PG-carrying bodies, it is preferred to administer, for each dose, from about 0.1-50 ml of liquid, containing an amount of PG-carrying bodies generally equivalent to 10%-1000% of the number of leukocytes normally found in an equivalent volume of whole blood or the number of apoptotic bodies that can be generated from them. Generally, the number of PG-carrying bodies administered per delivery to a patient is in the range from about 500 to about 2.5×10¹² (about 260 nanograms by weight), preferably from about 5,000 to about 500,000,000, more preferably from about 10,000 to about 10,000,000, and most preferably from about 200,000 to about 2,000,00

According to one feature of the invention, the number of such bodies administered to an injection site for each administration is believed to be a more meaningful quantization than the number or weight of PG-carrying bodies per unit of patient body weight. Thus, it is contemplated that effective amounts or numbers of PG-carrying bodies for small animal use may not directly translate into effective amounts for larger mammals on a weight ratio basis.

It is contemplated that the PG-carrying bodies may be freeze-dried or lyophilized to a form which may be later resuspended for administration. This invention therefore also includes a kit of parts comprising lyophilized or freeze-dried PG-carrying bodies and a pharmaceutically acceptable carrier, such as physiological sterile saline, sterile water, pyrogen-free water, isotonic saline, and phosphate buffer solutions, as well as other non-toxic compatible substances used in pharmaceutical formulations. Such a kit may optionally provide injection or administration means for administering the composition to a subject.

Since the pharmaceutically acceptable bodies are acting, in the process of the invention, as immune system modifiers, in the nature of a vaccine, the number of such bodies administered to an injection site for each administration maybe a more meaningful quantitation than the number or weight of bodies per unit of patient body weight. For the same reason, it is now contemplated that effective amounts or numbers of bodies for small animal use may not directly translate into effective amounts for larger mammals (i.e. greater than 5 kg) on a weight ratio basis.

4. Utility

The present invention is indicated for use in prophylaxis and/or treatment of a wide variety of mammalian disorders where T-cell function, inflammation, endothelial dysfunction and inappropriate cytokine expression are involved. A patient having or suspected of having such a disorder may be selected for treatment.

With respect to disorders involving inappropriate cytokine expression for which the present invention is indicated, these include any and all disorders involving inappropriate cytokine expression and include, for example, neurodegenerative diseases. Neurodegenerative diseases, including Down's syndrome, Alzheimer's disease and Parkinson's disease, are associated with increased levels of certain cytokines, including interleukin-1β (IL-1β) (see Griffin WST et al. (1989); Mogi M. et al. (1996)). It has also been shown that IL-1β inhibits long-term potentiation in the hippocampus (Murray, C. A. et al. (1998)). Long-term potentiation in the hippocampus is a form of synaptic plasticity and is generally considered to be an appropriate model for memory and learning (Bliss, T. V. P. et al. (1993)). Thus, inappropriate cytokine expression in the brain is currently believed to be involved in the development and progression of neurodegenerative diseases and neuroinflammatory disorders.

Parkinson's disease (PD) is an age related neurodegenerative disorder resulting at least in part from the degeneration of nigrostriatal dopaminergic neurons so that insufficient dopamine is generated in the brain. The disease has progressed to an advanced stage before clinical symptoms become evident, making it extremely difficult to attack early onset of the disease.

Inflammatory processes in the brain, for example proliferation and activation of microglia to induce release of pro-inflammatory cytotoxic factors including interleukin-1β (IL-1β) and tumor necrosis factor alpha (TNF-α) are associated with the neuropathology of PD. The substantia nigra, where dopaminergic neurons abound, is particularly enriched in microglia. Cytotoxic factors such as TNF a have a deleterious effect on neuronal survival. Thus, dopaminergic neurons in the substantia nigra are particularly sensitive to microglial activation.

Thus, the invention is indicated for the treatment and prophylaxis of a wide variety of mammalian neurodegenerative and other neurological disorders, including Downs syndrome, Alzheimer's disease, Parkinson's disease, senile dementia, depression, Huntingdon's disease, peripheral neuropathies, Guillain Barr syndrome, spinal cord diseases, neuropathic joint diseases, chronic inflammatory demyelinating disease, neuropathies including mononeuropathy, polyneuropathy, symmetrical distal sensory neuropathy, neuromuscular junction disorders, myasthenias and amyotrophic lateral sclerosis (ALS). Treatment and prophylaxis of these neurodegenerative diseases represents a particularly preferred embodiment of the invention, with treatment of Alzheimer's disease, Parkinson's disease and ALS particularly preferred.

With respect to T-cell function (T-cell mediated) disorders, these disorders include any and all disorders mediated at least in party by T-cells and include for example, ulcers, wounds, and autoimmune disorders including, but not limited to diabetes, scleroderma, psoriasis and rheumatoid arthritis.

The invention is indicated for use with inflammatory allergic reactions, organ and cell transplantation reaction disorders, and microbial infections giving rise to inflammatory reactions. It is also indicated for use in prophylaxis against oxidative stress and/or ischemia reperfusion injury, ingestion of poisons, exposure to toxic chemicals, radiation damage, and exposure to airborne and water-borne irritant substances, etc., which cause damaging inflammation. It is also indicated for inflammatory, allergic and T-cell-mediated disorders of internal organs such as kidney, liver, heart, etc.

Regarding disorders involving endothelial dysfunction, the present invention is indicated for the treatment and prophylaxis of a wide variety of such mammalian disorders including, any and all disorders mediated at least in part by endothelial dysfunction and include, for example, cardiovascular diseases, such as atherosclerosis, peripheral arterial or arterial occlusive disease, congestive heart failure, cerebrovascular disease (stroke), myocardial infarction, angina, hypertension, etc., vasospastic disorders such as Raynaud's disease, cardiac syndrome X, migraine etc., and the damage resulting from ischemia (ischemic injury or ischemia-reperfusion injury). In summary, it can be substantially any disorder the pathology of which involves an inappropriately functioning endothelium.

Further indications for the compositions and processes of the present invention include the treatment of patients to accelerate their rate of wound healing and ulcer healing, and treatment of patients prior to surgical operations, to accelerate their rate of recovery from surgery including their rate of healing of surgical wounds and incisions.

In regard to “cardiac disorders,” the present invention is indicated for the treatment and prophylaxis of a wide variety of such mammalian disorders including, any and all disorders relating to the heart and include, for example, ventricular arrhythmias (ventricular tachycardia or fibrillation) and sudden death from heart disease. Susceptibility of patients to cardiac disorders such as arrhythmias and sudden cardiac death is often indicated by prolonged QT-c intervals in the heart beat rhythm. Administration of compositions according to the preferred embodiments of the invention is believed to reduce QT-c intervals in mammalian patients, indicative of reduced susceptibility of to arrhythmia and sudden cardiac death.

The invention is further described, for illustrative purposes, in the following non-limiting examples.

### EXAMPLES

In the examples below, the following abbreviations have the following meanings. If an abbreviation is not defined, it has it generally acceptable meaning.
μg=microgram
μL=microliter
μm=micrometer
μM=micromolar
CHS=contact hypersensitivity
cm=centimeter
DMSO=dimethylsulfoxide
DNFB=2,4-dinitrofluorobenzene
DHS=delayed-type hypersensitivity
EtOH=ethanol
g=gram
hrs=hours
Hz=hertz
IM=intramuscular
IP=intraperitoneal
kg=kilogram
LPS=lipopolysaccharide
LTP=long-term potentiation
mg=milligram
min=minutes
ml=milliliter
mM=millimolar
ms=millisecond
ng=nanogram
nm=nanometer
nM=nanomolar
PBS=phosphate-buffered saline
PCR=polymerase chain reaction
POPS=1-palmitoyl-2-oleoyl-sn-glycero-3-[phospho-L-serine], referred to in the examples herein as PS
POPG=1-palmitoyl-2-oleoyl-sn-glycero-3-[phospho-rac-(1-glycerol)]], referred to in the examples herein as PG
POPC=1-palmitoyl-2-oleoyl-sn-glycero-3-phosphocholine, referred to in the examples herein as PC
RPM=revolutions per minute
S=second

Unless otherwise stated, the precise form of the lipids used in the experiments was POPS, POPG and POPC as set out above.

### Example 1

Liposomes of 100±20 nm in average diameter were prepared according to standard methods known in the art and had the following compositions:
Group A—100% PS
Group B—100% PG
Group C—control, no liposomes.

A stock suspension of each liposome composition containing 4.8×10¹⁴ liposomes per ml was diluted with PBS to give an injection suspension containing 6×10⁶ particles per ml. The liposomal suspensions were injected into female BALB/c mice (Jackson Laboratories) aged 6-8 weeks and weighing 19-23 g, to determine the effect on ear swelling in the murine contact hypersensitivity (CHS) model. The CHS model tests for Th1-mediated inflammatory reactions.

The animals were assigned to one of 3 groups, with 5 animals in each group. Groups A and B received approximately 3×10⁵ of the above-identified liposomes (i.e., 100% PC and 100% PG, respectively), in a volume of approximately 50 μl. Group C was a control group, receiving no liposomes.

### Protocol

The following experiments were performed:

| | Lipo- | | | | | | | Day 7 |
|---|---|---|---|---|---|---|---|---|
| Group | somes | Day 1 | Day 2 | Day 3 | Day 4 | Day 5 | Day 6 | (24 hours) |
| A | 100% | Injected | Injected | Injected | Injected | Injected | Injected | Ear |
| | PS | then | | | | | then | measured |
| | | sensitized | | | | | challenged | |
| B | 100% | Injected | Injected | Injected | Injected | Injected | Injected | Ear |
| | PG | then | | | | | then | measured |
| | | sensitized | | | | | challenged | |

On Days 1-6, mice of Groups A and B were injected with the respective liposomes preparations. Approximately 300,000 liposomes were injected in 50 μl volume via intramuscular (IM) injection, for a total administration over the test period of about 1,800,000 liposomes. Mice of the control group (Group C) received no liposomes, but were sensitized, challenged and tested in the same way as Groups A and B, as described below.

### Sensitization

On Day 1, following liposome injection for that day, mice were anaesthetized with 0.2 ml 5 mg/ml sodium pentobarbital via IP injection. The abdominal skin of the mouse was sprayed with 70% EtOH and a scalpel blade was used to remove about a one-inch diameter patch of hair from the abdomen. The shaved area was then painted with 25 μl of 0.5% 2,4-dinitrofluorobenzene (DNFB) in 4:1 acetone:olive oil using a pipette tip.

### Challenge

Following liposome injection on day 6, mice were challenged with DNFB by painting 10 μl of 0.2% DNFB on the dorsal surface of the right ear with a pipette tip and by painting 10 μl of vehicle on the left ear with a pipette tip.

### Results

On Day 7, 24 hours after challenge, each animal was anaesthetized with Halothane, and ear thickness was measured using a Peacock spring-loaded micrometer. Data was expressed as the difference between the treated right ear thickness and the thickness of the vehicle-treated left ear. The experiments were repeated three times, on similar animals. Increase in ear swelling was used as a measure of CHS response. The significance of the data was determined by the two-tailed student's t-test. A P value of <0.05 was considered significant.

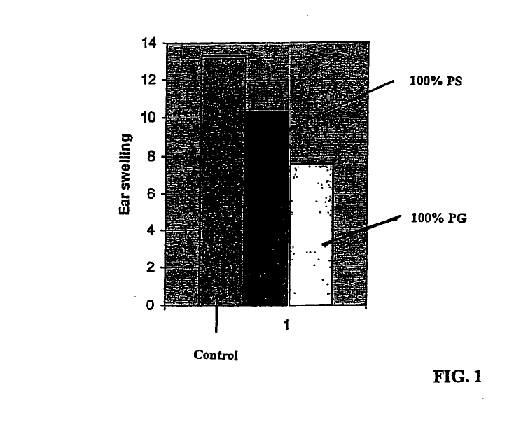

The results are presented in FIG. 1, a bar graph showing the mean values from the three experiments of ear swelling, reported in μm.

FIG. 1 shows that a significant reduction in ear swelling was achieved by injection of liposomes according to the present invention. The reduction achieved with 100% PG liposomes is substantially greater than that from 100% PS liposomes.

### Example 2

Liposomes of 100±20 nm in average diameter were prepared according to standard methods known in the art and had the following compositions:
Group A—100% PG
Group B—75% PG, 25% PC
Group C—50% PG, 50% PC
Group D—25% PG, 75% PC
Group E—PBS only
Group F—no injection

A stock suspension of each liposome containing 4.8×10¹⁴ liposomes per ml was diluted to give an injection suspension containing 12×10⁶ liposomes per ml. The liposomal suspensions were used to inject into mice to determine the effect on ear swelling in the murine CHS model, a biological system useful for assaying Th1-mediated inflammatory reactions. For these experiments, female BALB/c mice (Jackson Laboratories) aged 6-8 weeks and weighing 19-23 g were used.

The animals were assigned to one of 6 groups (Groups A-F, above) with 10 animals in each group. Control groups were also included that received no injections (Group F) or injections of PBS with no liposomes (Group E). Animals in Groups A-D were injected with 50 μl of the above-identified liposome suspensions, each containing about 6×10⁵ liposomes.

### Protocol

The test involves sensitization (Sens) with a potentially inflammation-causing substance, injection of liposomes (Inj) in test animals or PBS in controls and challenge (Chal) with the potentially inflammation-causing substance following measurement (Meas) to determine whether the injection of liposomes are effective against the development of inflammation by the challenge.

The following experiments were performed:

| Group | Lipo-somes | Day 1 | Day 2 | Day 3 | Day 4 | Day 5 | Day 6 | Day 7 |
|---|---|---|---|---|---|---|---|---|
| A | 100% PG | Sens & Inj | Inj | Inj | Inj | Inj | Chal & Inj | Meas |
| B | 75% PG | Sens & Inj | Inj | Inj | Inj | Inj | Chal & Inj | Meas |
| C | 50% PG | Sens & Inj | Inj | Inj | Inj | Inj | Chal & Inj | Meas |
| D | 25% PG | Sens & Inj | Inj | Inj | Inj | Inj | Chal & Inj | Meas |
| E | None | Sens & Inj | Inj | Inj | Inj | Inj | Chal & Inj | Meas |
| | (PBS only) | | | | | | | |
| F | none | Sens | | | | | Chal | Meas |

On days 1-6 the mice were injected with the respective liposomes as indicated above. Liposomes were injected in 50 μl volume via IM injection, i.e., 600,000 liposomes per injection, for a total administration over the test period of 3,600,000 liposomes. Mice of the control group received no liposomes but were sensitized, challenged and tested in the same way as the other groups of mice, as described below.

### Sensitization (Sens)

On Day 1, following liposome injection for that day, mice were anaesthetized with 0.2 ml 5 mg/ml sodium phenobarbital via IP injection. The abdominal skin of the mouse was sprayed with 70% EtOH and a blade was used to remove about a one inch diameter of hair from the abdomen. The bare area was painted with 25 μl of 0.5% 2,4-dinitrofluorobenzene (DNFB) in 4:1 acetone:olive oil using a pipette tip.

### Challenge (Chal)

On Day 6, following liposomes injection for that day, mice were challenged (Chal) with DNFB as follows: 10 μl of 0.2% DNFB was painted on the dorsal surface of the right ear with a pipette tip and 10 μl of vehicle was painted on the left ear with a pipette tip.

### Results

On Day 7, 24 hours after challenge, each animal was anaesthetized with Halothane, and ear thickness was measured (Meas) using a Peacock spring-loaded micrometer. Increase in ear swelling was used as a measure of CHS response. Data was expressed as the difference in the treated right ear thickness minus the thickness of the vehicle treated left ear. The significance between the two groups is determined by a two-tailed student's t-test. A P value of <0.05 is considered significant.

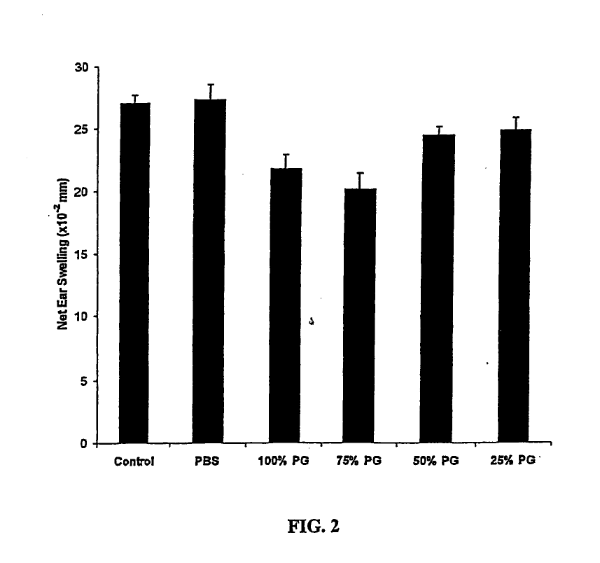

The results are presented graphically in FIG. 2, a bar graph showing ear swelling in μm. The mean value from the respective experiments was used in compiling the graph.

FIG. 2 shows that a significant reduction in ear swelling with both 100 and 75% PG is achieved, showing that both these concentrations protect against the development of inflammation resulting from contact with the allergenic substance, DNFB. The 50% and the 25% PG liposomes also showed reductions as compared with both controls, but the differences did not reach statistical significance in this experiment.

### Example 3

Liposomes of 100±20 nm in average diameter were prepared according to standard methods known in the art and were composed of 75% PG, 25% PC. A stock suspension containing 4.8×10¹⁴ liposomes per ml was used as before and diluted in PBS to give an injection suspension containing the following concentrations of liposomes:

| | | Concentration | Liposomes | |
|---|---|---|---|---|
| | | (liposomes per | per | Animals in |
| Group | Liposomes | mL) | injection | Group |
| A | 75% PG, | 12 × 10¹¹ | 6 × 10¹⁰ | 10 |
| | 25% PC | | | |
| B | 75% PG, | 12 × 10⁹ | 6 × 10⁸ | 10 |
| | 25% PC | | | |
| C | 75% PG, | 12 × 10⁸ | 6 × 10⁷ | 16 |
| | 25% PC | | | |
| D | 75% PG, | 12 × 10⁷ | 6 × 10⁶ | 16 |
| | 25% PC | | | |
| E | 75% PG, | 12 × 10⁶ | 6 × 10⁵ | 16 |
| | 25% PC | | | |
| F | none (PBS | | | 16 |
| | only) | | | |

BALB-c mice were divided into six groups (Groups A-F) including a control group receiving no liposomes but injected with 50 μL of PBS (Group F). Mice were sensitized on the flank, injected with their selected liposomal dose, intramuscularly to the right leg muscle, on the same day as, but after, sensitisation (day 1) and on days 2, 3, 4, and 5. On day 6 they were both injected and challenged on the ear as described in Example 1. The thickness of the ear was measured as described 24 hours after the challenge.

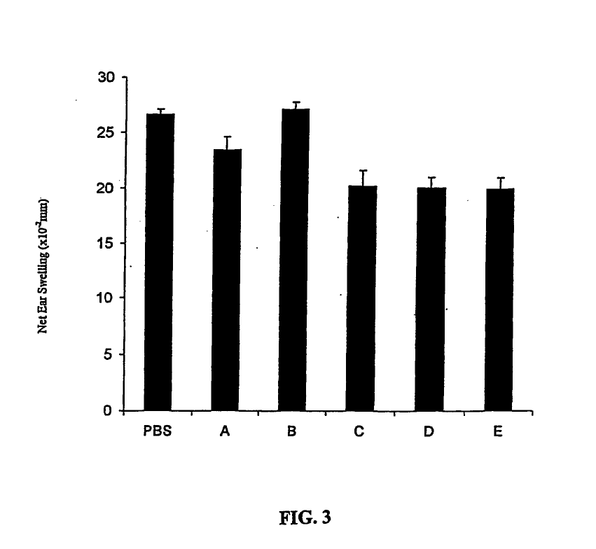

The results (FIG. 3) show a significant difference between the control group (Group F) and Group C (12×10⁸ liposomes per ml) and between the control group and Group D (12×10⁷ liposomes per ml) and between the control group and Group E (12×10⁶ liposomes per ml). There was little difference between the control group and Groups A or B (12×10¹¹ and 12×10⁹ liposomes per ml, respectively), suggesting that there is an optimum range of liposome concentrations above which the beneficial effects may be reduced. In other experiments, a decrease in effect was also be observed as the concentration of the liposomes was decreased below 12×10⁴ liposomes per ml.

### Example 4

Liposomes of formulation 100% PG and 100±20 nm in average size were prepared according to standard methods. Four groups (Groups A-D) of 10 mice were sensitised, injected and challenged in accordance with the procedure and schedule described in Example 3, with the following numbers of 100% PG liposomes delivered in a 50 μl suspension.
Group A—6×107
Group B—6×106
Group C—6×105
Group D—6×104

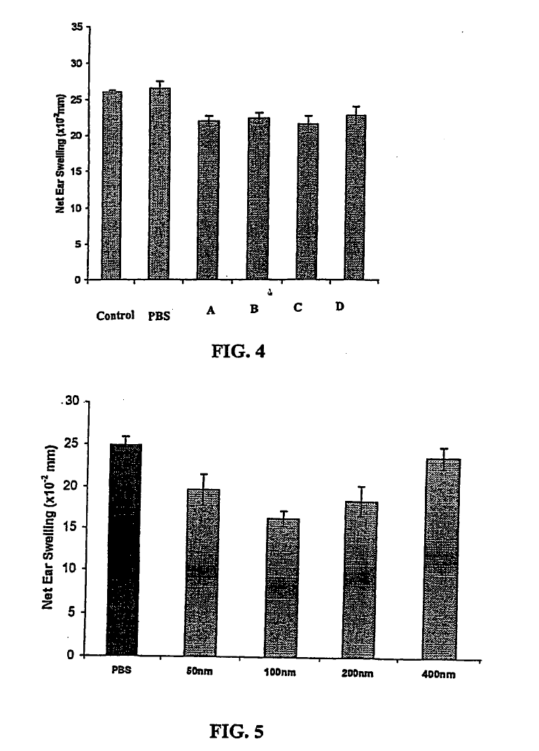

The results, along with the PBS control from Example 4, are presented in similar bar graph form in FIG. 4. A significant reduction in ear swelling, as compared with the control group is to be noted for each of the test groups, but with little difference between the various groups.

### Example 5

Liposomes of composition 75% PG, 25% PC and of 50, 100, 200, of 400 nm in average diameter were prepared by standard methods. They were tested in the murine CHS model, as in Examples 3 and 4, using 6×10⁵ liposomes in 50 μl suspensions for each injection, and a sensitisation-injection-challenge schedule and procedure as in Example 3. The groups were as follows:

| | |
|---|---|
| Group A | 50 nm liposomes |
| Group B | 100 nm liposomes |
| Group C | 200 nm liposomes |
| Group D | 400 nm liposomes |
| Group E | no liposomes |

The results are presented in FIG. 5. The result from Group D, using the 400 nm diameter liposomes, is not significantly different from the control group (Group E), indicating a probable size range criticality in this model.

### Example 6

A stock suspension of 75% PG liposomes of 100±20 run in average diameter containing 4.8×10¹⁴ liposomes per ml was diluted to give an injection suspension containing 6×10⁵ liposomes per ml. The liposomal suspensions were used to inject into mice, to determine the effect on ear swelling in the murine DHS model. As in Example 1, female BALB/c mice (Jackson Laboratories) aged 6-8 weeks and weighing 19-23 g were used.

The animals were assigned to one of 3 groups with 10 animals in each group. A control group (Group C) received only PBS injections. Animals of Groups A and B were injected with 50 μl of a suspension containing 6×105 liposomes.

### Protocol

On days 13-18 the mice were injected with the 75% PG liposomes as indicated below. Liposomes were injected in 50 μl volume via IM injection, i.e., 600,000 liposomes per injection, for a total administration over the test period of 3,600,000 liposomes. Sensitization and challenge took place as described in Example 2.

| DAY | TREATMENT |
|---|---|
| 1 | Sensitized |
| 6 | Challenged |
| 7 | Measured |
| 12 | Challenged |
| 13 | Measured & Injected |
| 14 | Injected |
| 15 | Injected |
| 16 | Measured & Injected |
| 17 | Injected |
| 18 | Injected & Challenged |
| 19 | Measured |

### Results

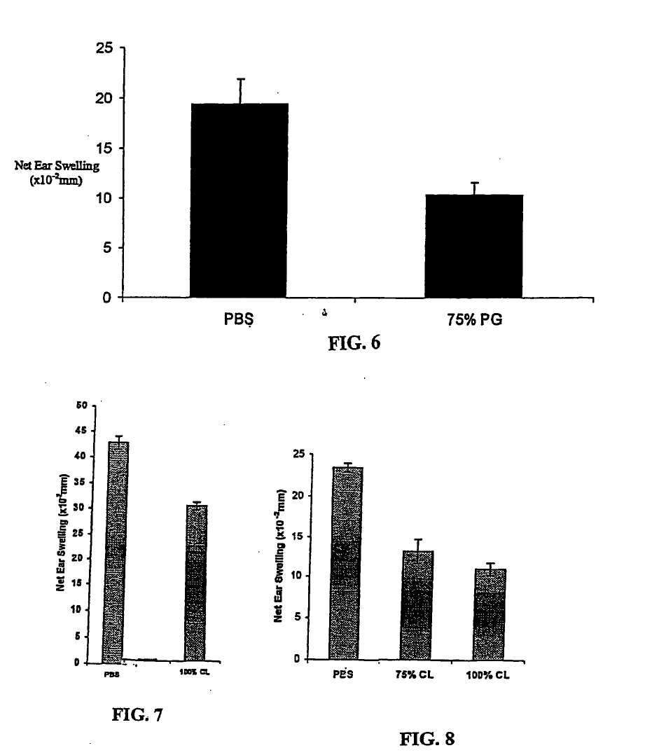

The results are presented graphically in accompanying FIG. 6 and show that 75% PG is effective in the DHS model on day 16, 24 hours after the third injection following the second challenge.

### Example 7

Liposomes of composed of 100% cardiolipin (CL) and 100±20 nm in average diameter were prepared, by standard methods. These were used at a dosage of 6×10⁵ liposomes per 50 μl per injection in the murine DHS model described in Example 6. Data obtained from animals injected with CL liposomes (Group A; 10 animals) was compared to data obtained from animals receiving only PBS (Group B; 10 animals). The sensitization, injection and challenge procedures were as described in Example 2. The ear thickness measurement results, taken on day 19, 24 hrs after the 6th injection, are presented in FIG. 7. The results showed a significant reduction in ear swelling within the CL-injected test (Group A).

### Example 8

Liposomes of 100 nm in average diameter, and comprising either 100% cardiolipin or 75% cardiolipin and 25% PC, were prepared by standard methods. Three groups (Groups A-C) of 10 mice were sensitized on day 1. A control group received injections of PBS on days 1, 2 and 6 (Group C). The other two groups received injections, of 6×10⁵ 100% cardiolipin liposomes (Group A) or of 6×10⁵ 75% cardiolipin liposomes (Group B), liposomes in 50 μl per injection according to the same schedule. The mice were challenged on day 7, and the ear thickness measured, as described in the previous examples.

FIG. 8 shows the mean measurements in each group. Both groups receiving CL liposomes showed a statistically significant suppression of CHS compared to the control group

### Example 9

To study the cellular and molecular mechanisms underlying cognitive function, the Long-Term Potentiation (LTP) animal model is used. LTP is a form of synaptic plasticity that occurs in the hippocampal formation, which has been proposed as a biological substrate for learning and memory (Bliss, et al. Nature 361:31-39 (1990)). LTP in rats is monitored electrophysiologically by methods well known to those in the art. The animals are then sacrificed to investigate biochemical changes in hippocampal tissues. Comparing the results of electrophysiological data with biochemical hippocampal changes is useful for determining how the cellular events that underlie LTP may be altered in animals suffering from diseases or disorders associated with neuroinflammation such as aging, stress, Alzheimer's disease, and bacterial infection.

Systemic administration of lipopolysaccharide (LPS), a cell-wall component of Gram-negative bacteria, provokes an activation of the immune system by inducing an increase in pro-inflammatory cytokines such as IL-1β. As noted above, one example of a neuronal deficit induced by LPS and IL-1β is the impairment of LTP in the hippocampus. An indicator of LTP is the mean slope of the population excitatory post-synaptic potential (epsp). Upon tetanic stimulation, the epsp slope (%) increases sharply indicating increased synaptic activity. LPS-induced inhibition of LTP reduces the increase in slope, and/or causes the epsp slope to revert more rapidly to base line, indicating that the increased synaptic activity is short-lived. Accordingly measurements of the epsp slope (%) at timed intervals after tetanic stimulation can be used to reflect memory and the loss thereof following an inflammatory stimulus as well as inflammation in the hippocampus of the brain.

Liposomes of 100±20 nm in average diameter were prepared as according to standard methods known in the art and were composed of 75% PG and 25% PC. A stock suspension of the liposomes containing about 2.9×10¹⁴ liposomes per ml was diluted with PBS to give an injection suspension containing about 1.2×10⁷ liposomes per ml. This was then used to inject into rats, to determine the effect on LPS-induced impairment of LTP. For these experiments, male Wistar rats (BioResources Unit, Trinity College, Dublin), weighing approximately 300 g, were used.

The animals were assigned to one of four groups, 8 animals in each group to be treated as follows:

| | |
|---|---|
| Group A | saline + control |
| Group B | saline + PG |
| Group C | LPS + control |
| Group D | LPS + PG |

150 μl of each above-identified preparation was injected via IM injection on days 1, 13, and 14. Groups B and D received a total of 5,400,000 liposomes (1,800,000 liposomes per injection). The LTP procedure and tissue preparation procedure were carried out on day 0.

### LTP Procedure

Rats were anaesthetized by IP injection of urethane (1.5 g/kg). Rats received either LPS (100 μg/kg) or saline intraperitoneally. Three hours later a bipolar stimulating electrode and a unipolar recording electrode were placed in the perforant path and in the dorsal cell body region of the dentate gyrus respectively. Test shocks of 0.033 Hz were given and responses recorded for 10 min before and 45 min after high frequency stimulation (3 trains of stimuli delivered at 30 s intervals, 250 Hz for 200 ms).

Rats were killed by decapitation. The hippocampus, the tetanized and untetanized dentate gyri, the cortex and entorhinal cortex were dissected on ice, sectioned and frozen in 1 ml of Krebs solution (composition of Krebs in mM: NaCl 136, KCl 2.54, KH₂PO₄ 1.18, MgSO₄.7H₂O 1.18, NaHCO₃ 16, glucose 10, CaCl₂ 1.13) containing 10% DMSO.

### Results

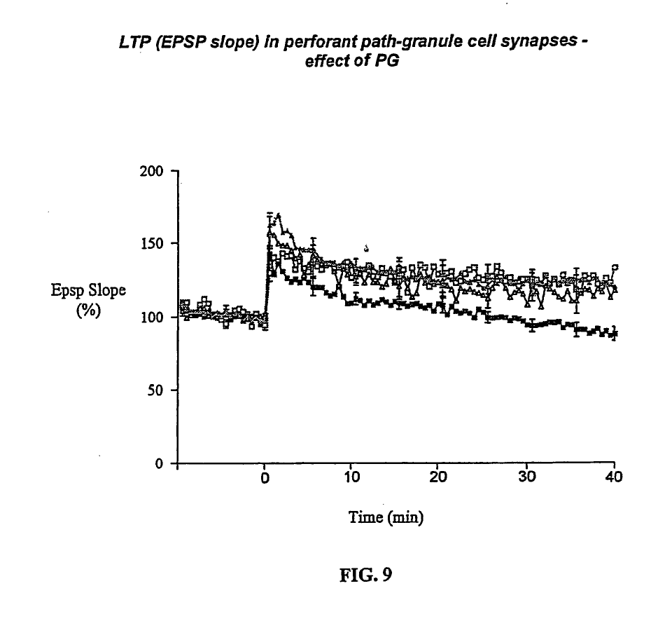

The results are shown in FIG. 9. The graph shows the difference in the excitatory post-synaptic potential (epsp) recorded in cell bodies of the granule cells. The data presented are means of seven to eight observations in each treatment group and are expressed as mean percentage change in epsp slope every 30 s normalized with respect to the mean value in the 5 minutes immediately prior to tetanic stimulation. FIG. 9 shows that the LPS-induced inhibition of LTP in perforant path-granule cell synapses was overcome by pre-treatment with the PG liposomes. The filled triangles represent Group A (saline+control), the open triangles represent Group B (saline+PG), the filled squares represent Group C (LPS+control) and the open squares represent Group D (LPS+PG).

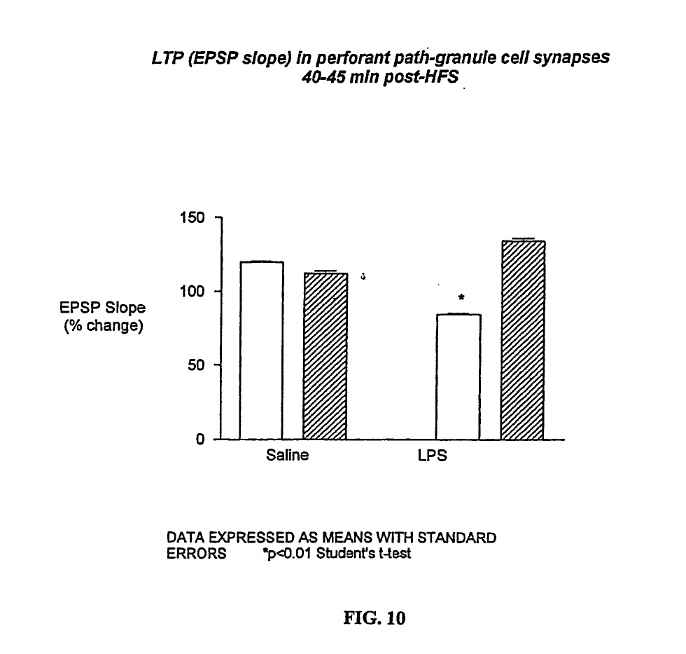

FIG. 10 shows that analysis of the mean values 40-45 minutes post tetanic stimulation indicate that the population epsp slope was decreased in the control-LPS group (open bars) and that the PG liposomes (hashed bars) significantly reversed this effect (*p<0.01). As an index of memory and learning functionality, the improvement in sustainability of LTP demonstrated in this Example indicates suitability of the treatment for dementias e.g. Alzheimer's disease and memory impairment.

### Example 10

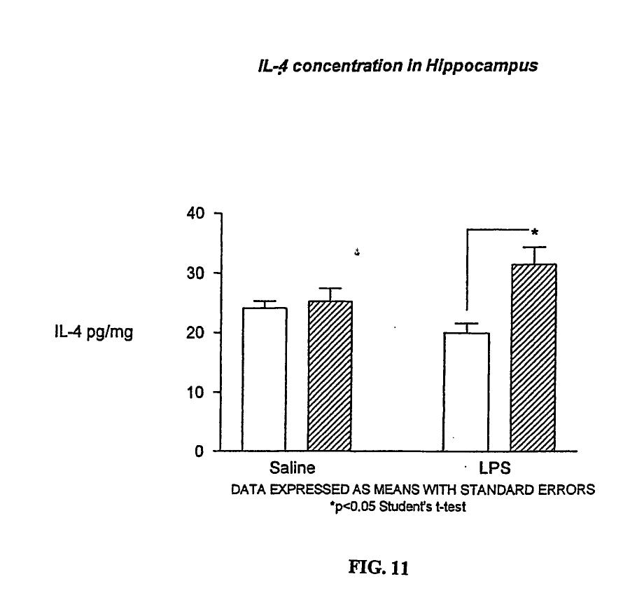

IL-4 is one of a number of cytokines secreted by the Th2 subclass of lymphocytes and is known for its anti-inflammatory effects. FIG. 11 shows that the IL-4 concentration in the hippocampus was significantly increased in the LPS group that had been pre-treated with the PG liposomes (*p<0.05). Open bars represent control group (Group E) and hashed bars represent the PG treated group (Group F). IL-4 was measured by ELISA and expressed as PG of IL-4 per mg of total protein.

This upregulation of the anti-inflammatory cytokine IL-4 in the brain is indicative of the use of the process and composition of preferred embodiments of the present invention in treating a wide range of neuroinflammatory disorders, including Parkinson's disease, ALS, chronic inflammatory demyelinating disease CIPD and Guillain Barr syndrome.

### Example 11

IL-1β is one of a number of cytokines secreted by the Th1 subclass of lymphocytes and is known for its proinflammatory effects. Spleens from animals treated as described in Example 9, groups C and D thereof, were extracted and spleen cells collected.

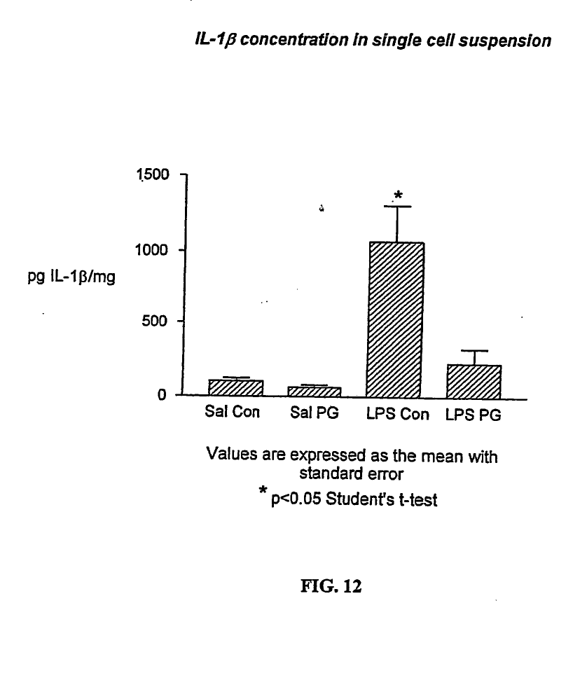

FIG. 12 shows that the IL-1β concentration in spleen cells was significantly reduced in the LPS group that had been pre-treated with the PG liposomes (*p<0.05). IL-1β was measured by ELISA and expressed as picagrams of IL-1β per mg of total protein. This indicates a systemic inflammatory effect of the process and compositions of preferred embodiments of the present invention.

### Example 12

U937 is a monocytic leukemia cell line that can be differentiated into macrophages by administration of phorbol esters. Treatment with lipopolysaccharide (LPS), a component of the cell wall of Gram-negative bacteria, stimulates an inflammatory response in U937 cells, with the upregulation of expression of a number of inflammatory molecules including TNFα. This model provides an experimental system for the assessment of anti-inflammatory therapies. The macrophages can be grown in culture medium in the presence of a suspected anti-inflammatory composition, and the expression of TNFα can be measured.

Liposomes of 100±20 nm in average diameter were prepared according to standard methods known in the art and had a composition of 75% phosphatidylglycerol (PG), 25% phosphatidylcholine (PC). The stock concentration of liposome was about 40 mM lipid and was diluted to the following final concentrations in the assay:
100 μM phosphatidylglycerol (PG)
40 μM PG
10 μM PG
4.0 μM PG
1 μM PG

The U937 cells were cultured by growing in RPMI medium (GIBCO BRL) supplemented with 10% fetal bovine serum (FBS) and 1% penicillin/streptomycin and grown at 37° C. in an atmosphere containing 5% CO₂.5×10⁵ cells were seeded into wells of 6-well plates and caused to differentiated into macrophages by treatment with 150 nM phorbol myristate acetate (PMA) for 2-3 days. The cell medium was then replaced with complete medium after the U937 cells had differentiated into macrophages. The cells were then incubated for an additional 24 hrs to minimize pleotropic effects due to PMA treatment.

The cells were then incubated with either:

| | |
|---|---|
| Group A | Phosphate buffered saline (PBS) - as a negative control, |
| Group B | 10 ng/ml LPS - as a positive control, |
| Group C | 10 ng/ml LPS + 100 μM PG, |
| Group D | 10 ng/ml LPS + 40 μM PG, |
| Group E | 10 ng/ml LPS + 10 μM PG, |
| Group F | 10 ng/ml LPS + 4.0 μM PG, or |
| Group G | 10 ng/ml LPS + 1 μM PG. |

The cells were incubated as described above at 37° C. in 5% CO₂. After 18 hrs, the supernatants from each treatment were collected and assayed for TNF-α using a standard Quantikine Enzyme-Linked Immunosorbent Assay (ELISA) kit (R&D systems, Minneapolis, USA).

### Results

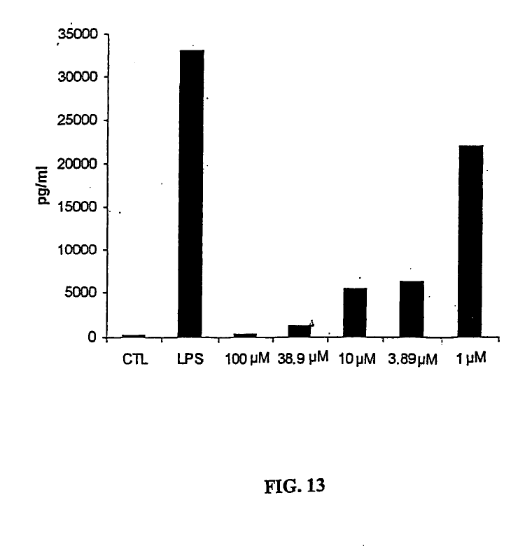

FIG. 13 shows the amount of secreted TNF-α in PG per ml. The results demonstrates that U937-differentiated macrophage cells express very low levels of TNF-α under normal conditions. However, once exposed to LPS, they secrete large amounts of TNF-α into the surrounding medium, which is indicative of cellular stress occurring. Incubation of the cells with PG liposomes inhibits the secretion of TNF-α in a dose-dependent manner, with the highest concentration of 100 μM resulting in a 98% decrease, and even the lowest concentration of 1 μM causing a 58% decrease in TNF-α expression.

### Example 13

To determine the effect of the PG liposomes of the preferred embodiment of the present invention on endothelial function, the endothelin-1 (ET-1) content in the ears of mice which had been subjected to the CHS studies as described in Example 3 was determined. Endothelin-1 is a potent vasoconstrictive agent, has inotropic and mitogenic actions, modulates salt and water homeostasis and plays an important role in the maintenance of vascular tone and blood pressure. Various lines of evidence indicate that endogenous ET-1 may contribute to the pathophysiology of conditions associated with sustained vasoconstriction, such as heart failure. In heart failure, elevated levels of circulating ET-1 and big-ET-1 are observed (Giannessi D, et al., “The role of endothelins and their receptors in heart failure.” Pharmacol. Res. (2001) 43:2 111-26). Thus ET-1 is a marker of endothelial function and increased production of ET-1 in tissue is indicative of impaired endothelial function.

In order to determine ET-1 expression, mouse ears (right challenged ear) were harvested 24 hrs after challenge in CHS experiments. Ears were obtained from mice injected intramuscularly with PBS for 6 days (Group A) and mice injected intramuscularly with 75% PG/25% PC liposomes (600,000 liposomes/injection; Group B). Ears were stored in RNAlater at −20° C. until RNA extraction. RNA was extracted and cDNA was generated using reverse transcriptase (RT) along with ET-1-specific primers, as an internal control, PCR was also performed using 3-actin-specific primers. PCR products were resolved on a 1.5% agarose gel and the DNA bands were quantitated by densitometry analysis. The ratio of ET-1/β-actin was calculated.

PCR Preparation:

| | |
|---|---|
| PCR Mix (ET-1) | PCR Mix (β-Actin) |

| | | | |
|---|---|---|---|
| 5 μl | PCR Buffer (10×) | 5 μl | PCR Buffer (10×) |
| 1.5 μl | MgCl2 (50 mM) | 1.5 μl | MgCl2 (50 mM) |
| 1 μl | dNTP (10 mM) | 1 μl | dNTP (10 mM) |
| 0.5 μl | Primer 1 (25 uM) | 1 μl | Primer 1 (10 uM) |
| 0.5 μl | Primer 2 (25 uM) | 1 μl | Primer 2 (10 uM) |
| 0.25 μl | TAQ | 0.25 μl | TAQ |
| 2.5 μl | cDNA | 2.5 μl | cDNA |
| 38 μl | Water | 37.75 μl | Water |
| 50 μl | Total | 50 μl | Total |

Primers: (as previously described in Yang, et al. “Conditional cardiac overexpression of endothelin-1 in transgenic mice,” FASEB J. 15(5): A1138-A1138 Part 2 (2001)).

| | |
|---|---|
| ET-1(r) | 5′-CAG CAC TTC TTG TCT TTT TGG-3′ |
| ET-1(f) | 5′-CCA AGG AGC TCC AGA AAC AG-3′ |
| β-Actin(F) | 5′-GTG GGC CGC TCT AGG CAC CAA-3′ |
| β-Actin(r) | 5′-CTC TTT GAT GTC ACG CAC GAT TTC-3′ |

PCR Settings:
94° C.—5 minutes

$$\begin{matrix}\begin{matrix}94 ° C . - 30 s \\ 60 ° C . - 30 s\end{matrix} \\ 72 ° C . - 60 s\end{matrix} \} 30 cycles$$
72° C.—10 minutes
4° C.—Soak

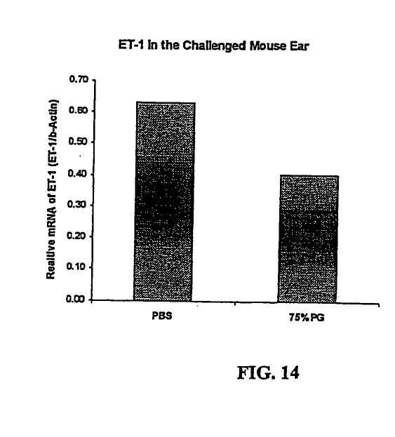

After 6-daily injections of the 75% PG liposomes, the level of ET-1 was decreased by 36% relative to control mice receiving PBS during the same injection regimen. The results are shown graphically on FIG. 14. This decrease indicates a beneficial effect resulting from the injection of the liposomes of the preferred embodiment of the invention on endothelial function in a mammalian patient, through Th1 mediated inflammation reduction.

### Example 14

Intercellular adhesion molecule-1 (ICAM-1) is a cell surface molecule expressed by several cell types, including leukocytes and endothelial cells. It is involved in the adhesion of monocytes to endothelial cells and plays a role in inflammatory processes and in the T-cell mediated host defense system. ICAM-1 expression probably contributes to the clinical manifestations of a variety of diseases, predominantly by interfering with normal immune function. Among these are malignancies (e.g., melanoma and lymphomas), many inflammatory disorders (e.g., asthma and autoimmune disorders), atherosclerosis, ischemia, certain neurological disorders, and allogeneic organ transplantation (Van de Stolpe A, van der Saag P T, “Intercellular adhesion molecule-1” J. Mol. Med. (1996) 74:1 13-33).

Human umbilical vein endothelial cells (HUVECs) are a primary cell line of endothelial cells that are isolated from umbilical vein cords as follows.

T75 flasks were prepared by coating with 0.2% gelatin (5-7 ml/flask) for a minimum of 15/20 minutes or overnight. The excess was then removed. The cord was sprayed with 70% ethanol prior to procedure and any placenta still remaining attached to the cord was cut away. The cord was then cut to an approximate length of 5-6 inches. The cord has two arteries which are thick walled and one vein that is bigger and thin walled. The vein was located and the serrated edge of a stopper placed into it. Approximately 20 cm of string was then used to tie the cord onto the stopper.

The cord was then washed through with phosphate buffered saline (PBS) a number of times until the PBS ran clear. Following this 15-20 ml of Collagenase solution was placed into the cord; it was wrapped in tinfoil and incubated for 15 minutes at 37° C. After incubation the tied end of the cord was cut and the collagenase drained into a 50 ml tube. Collagenase was then passed through the cord again, the cord was massaged to loosen the endothelial cells and then PBS was passed through the cord and collected into the same tube containing the collagenase solution. This was then centrifuged at 1600 RPMs, the supernatant removed and the pellet resuspended in 10-12 ml of M199 complete medium. Finally the medium containing the cells was added to the gelatinized flasks.

Liposomes of 100±20 nm in average diameter were prepared according to standard methods known in the art and had a composition of 75% phosphatidylglycerol (PG), 25% phosphatidylcholine (PC). The stock concentration of liposome was 40 mM lipid and was diluted to 100 μM in the assay.

HUVECs split into a number of tissue culture flasks, allowed to adhere to the surface of the flask and then treated as follows:
Group A—PBS—as a negative control,
Group B—500 ng/ml LPS—as a positive control,
Group C—500 ng/ml LPS+100 μM PG
Group D—500 ng/ml LPS+100 μM PC

The cells were incubated at 37° C., 5% CO₂. After 18 hrs, the supernatants from each treatment were collected and assayed for ET-1 using a standard ELISA kit (obtained from Assay Designs) and the cells harvested for analyzing ICAM-1 as follows.

The cells were first washed with PBS and then incubated with a cell dissociation buffer at 37° C. for 25-30 min. The cells were then washed by centrifugation and incubated with an anti-CD54 (ICAM-1) antibody for 30 minutes. A secondary FITC antibody was then added and incubated with the cells as before. Finally they were resuspended in 1 ml of PBS and analyzed for fluorescence on a flow cytometer.

### Results

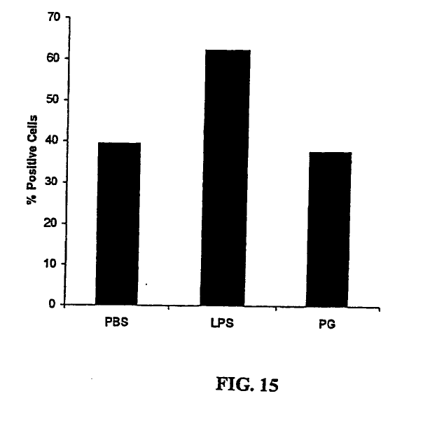

The results are presented on FIG. 15, a graphical presentation of the percentage of cells staining positive for ICAM-1 in the respective cultures. It is to be noted that the numbers of cells staining positive in the PG liposome-containing culture is reduced to negative control level, and is much lower than the positive control level.

### Example 15

Microglial cells (brain macrophages) were cultured, and their output of TNF-α, an inflammatory cytokine, was measured. The cells were stimulated with the immunoglobulin (IgG) of patients suffering from ALS, and the TNF-αc output increased about 800-fold as a result. When the same cells were grown in the presence of both the ALS IgG and PG liposomes, output of TNF-α decreased by about 75%, indicating the potential of the preferred embodiments of the present invention in the treatment of ALS.

### Example 16

Experimental models have been designed to mimic PD, in attempts to develop therapeutic strategies in its treatment. Many of these use the chemotoxin 6-hydroxydopamine (6-OHDA). When introduced into the cell bodies and nerve fibers of dopaminergic neurons, it exerts potent cytotoxic effects via inhibition of mitochondrial complexes. Intranigral or intrastriatal unilateral stereotaxic 6-OHDA injection in rodents produces a dramatic dropout of dopaminergic neurons in the substantia nigra pars compacta (SNpc) accompanied by a marked reduction of dopaminergic terminals in the striatum. Introduction of 6-OHDA into one hemisphere of the brain causes lesions in the SNpc in that hemisphere, leaving the SNpc in the other hemisphere intact. This imbalance between hemispheres causes a marked asymmetry in the motor behavior of the animals 4-7 days post lesion. The animals typically display an initial bias towards the side of the lesion. Subsequent intraperitoneal administration of the dopaminomimetic drug D-amphetamine creates a dopamine imbalance that favors the non-lesioned hemisphere and thus generates marked ipsilateral rotation (turns towards the lesioned hemisphere). The ability of a substance to counteract this rotational behavior in the animals so treated is an indicator of its potential to treat or guard against progression of PD.

### EXPERIMENTAL PROCEDURES

Groups of male Sprague-Dawley rats (225-250 grams, Biological Service Unit, University College Cork) were used in these experiments. Animals were maintained in the temperature and humidity controlled environment under the 12-hour light schedule with food and water available ad libitum. The rats were caged in groups of six during the presurgical period and then individually housed following the lesion. All animal procedures strictly adhered to local and national guidelines.

All rats were treated intramuscularly with phosphatidyl glycerol-containing liposomes as described in Examples 9 and 12 above; 150 μl of a 1.2×10⁷ particles/ml (Composition A) and 1.2×10¹⁰ particles/ml (Composition B) suspension in phosphate-buffered saline, or saline, 14 days, 13 days and 1 day before unilateral lesioning of the medial forebrain bundle with 6-OHDA; (8 μg/4 μl). Administration of either drug or control was alternated between the left and right hind limbs on alternate days in an attempt to minimize local muscle injury. Functional integrity of the nigrostriatal dopaminergic neurons was assessed one week before the surgery using an intraperitoneal D-Amphetamine challenge (5 mg/kg) in all animal groups. Following the surgery, rats were either sacrificed four days later, or exposed to a minimum of one further behavioral assessment. Following the final amphetamine challenge, rats were sacrificed by decapitation, and cortex was dissected from both hemispheres for preparation of homogenate (n=6 per group).

Two weeks after the initial exposure to either vehicle or liposomes, rats were anaesthetized with a 1:1 mixture of xylazine hydrochloride (Vetoquinol UK Ltd) and ketamine hydrochloride (Chassot, Dublin, Ireland) with 1.50 ml of each compound dissolved in 7 ml of PBS. An injection volume of 0.2 ml/100 g body weight provided adequate analgesia and ensured that the rats were at surgical plane for at least one hour. Depth of anaesthesia was assessed using pedal, tail and corneal reflex tests. Once sufficient anaesthesia was obtained the animals were prepared for surgery and placed into a stereotaxic frame (David Knopf Instruments, Tujunga, USA). Prior to electrode placement, ventral measurements were recorded for bregma and lambda to ensure that the skull was a level is possible while in the frame. Fine adjustments of the nose bar were made until differences in ventral coordinates between bregma and lambda were less than or equal to 0.1 mm.

A small burr hole was drilled in the skull at the following coordinates: AP-2.2 mm, ML+1.5 mm from bregma. A 10 μl Hamilton syringe partially filled with 6-OHDA hydrobromide (Sigma, UK) was then slowly lowered into the MFB (7.8 mm ventral from brain surface). All stereotaxic coordinates were in reference to the stereotaxic rat brain atlas of Paxinos and Watson, “The rat brain in stereotaxic coordinates”, Academic Press, California, USA 1998. Once the tip of the needle was in place the surrounding brain tissue was allowed sufficient time (˜5 minutes) to reform around the needle before infusion of the neurotoxin. The 6-OHDA was then slowly infused (0.5 μl/min) at a concentration of 2 μg/μl (free base) and the needle left in place to allow for complete diffusion of the 6-OHDA into the surrounding brain tissue. The needle was then slowly withdrawn and the animal sutured closed before receiving post-operative care until it recovered fully for the anaesthesia.

Sham surgery groups received the exact same surgical protocol with the notable exception of 4 μl of saline rather than 6-OHDA.

Selected groups of rats were challenged with 5 mg/kg D-amphetamine intraperitoneally 1 week (and in some cases again at 3 weeks) post-lesion. Once injected, the rats were individually placed into a cylinder with bedding at the base and continually monitored for at least 45 minutes. Rotational counts were measured every 5 minutes for a period of 1 hour. Once the effects of the amphetamine had worn off all animals were rehydrated with 3 ml saline and placed back into their holding cage.

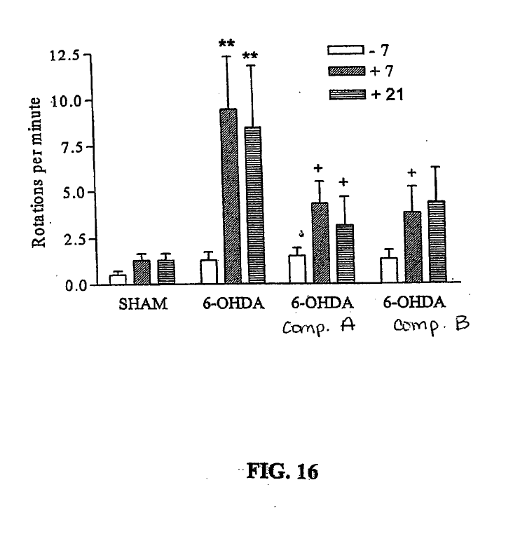

The results are presented graphically on accompanying FIG. 16. These are mean values of 8-10 animals per group. Rotations counted per minute are plotted on the vertical axis. The first three bars derive from animals which received sham (saline) injections and no 6-OHDA, and show insignificant rotational behaviour. The second three bars show animals treated with saline, followed by 6-OHDA administration, and challenged with D-amphetamine 7 days later (middle bar) and again 21 days after 6-OHDA administration (right bar). In both cases, the lesioned rats exhibited rotational behaviour, to the extent of about 8-9 rotations per minute. The third and fourth groups of three bars show, correspondingly, the behaviour of PG/PC-liposome treated rats. Statistical analysis using a 2-way analysis of variance (ANOVA) followed by Tukeys post hoc analysis shows a significant difference in the animals treated according to the invention, an indicator of potential anti-PD effects.

Animals were sacrificed at predetermined time points by decapitation and their brains rapidly removed. Cortical tissue from both hemispheres was microdissected out on ice and cross-chopped into slices (350×350 μm) using a McIlwain tissue chopper. Brain sections were placed into eppendorf tubes containing Krebs buffer with CaCl₂ (1.13 mM). The tissue was washed 3 times in Krebs buffer before being placed in a Krebs-Dimethyl Sulphoxide (10%) solution and stored at −80° C. as described by Haan and Bowen, 1981, J. Neurochem. 37, 243-246, until required for analysis.

TNF-α concentration in homogenate prepared from cortical tissue was analysed by enzyme-linked immunosorbent assay (ELISA; DuoSet; R&D Systems). Cortical slices were thawed, and rinsed three times in ice-cold Krebs solution and homogenized in ice-cold Krebs solution. Protein concentrations in homogenates were equalized and triplicate aliquots (100 μl) were used for ELISA. Antibody-coated (4.0 μg/ml mouse anti-rat TNF-α diluted in PBS, pH 7.3) 96-well plates were incubated overnight at 4° C., washed thoroughly with PBS containing 0.05% Tween 20, blocked for 1 h with 300 μl of blocking buffer (PBS, pH 7.3, with 1% bovine serum albumin), and incubated with standards (100 μl; 0-4000 pg/ml) or samples for 2 hours at room temperature. Samples were incubated with secondary antibody (100 ng/ml biotinylated goat anti-rat TNF-α in PBS containing 1% bovine serum albumin) for 2 h at room temperature. ELISA plates were then washed and incubated in detection agent (100 μl; horseradish peroxidase-conjugated streptavidin; 1:200 dilution in PBS contiaing 1% bovine serum albumin) in the dark for 20 min at room temperature. Substrate solution (1:1 mixture of H₂O₂ and tetramethylbenzidine; R&D Systems) was added, incubation continued at room temperature in the dark for 30 min and the reaction stopped using 1M H₂SO₄. Absorbance was read at 450 nm using a Sunrise microplate reader; values were corrected for protein in the case of homogenates and expressed as pg/mg protein.

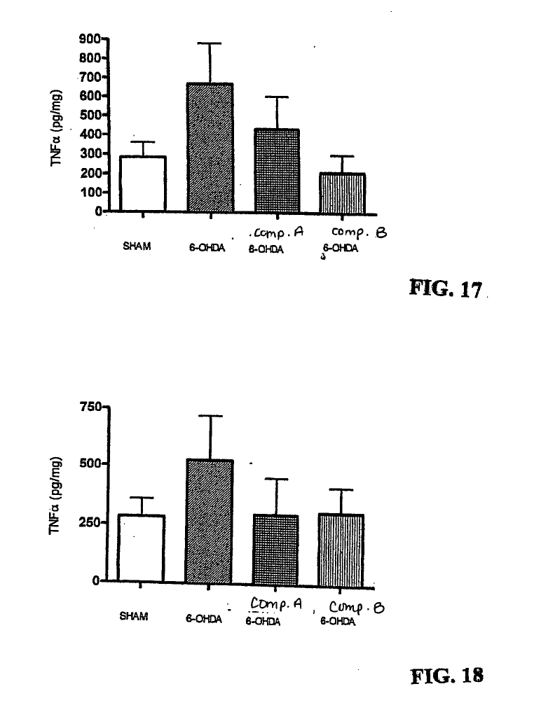

The results are presented graphically on the accompanying FIGS. 17 and 18. TNF-α is shown to be reduced in the cortex of the animals treated according to the invention, after 10 days (FIG. 17) and after 28 days (FIG. 18) from 6-OHDA administration, substantially down to control (sham treated) levels. The products of the preferred embodiment of the present invention can be concluded to have a TNF-α down regulation effect on at least part of the underlying mechanism of their anti-PD effects.

1. A composition of matter capable of producing an anti-inflammatory response in vivo in a mammal, said composition comprising pharmaceutically acceptable bodies of a size from about 20 nanometers (nm) to 500 micrometers (μm), comprising a plurality of phosphatidylglycerol (PG) head groups or groups convertible to PG head groups.

2. A method for the prophylaxis, treatment or delay in the progression of Parkinson's disease in a mammalian patient which comprises reducing the level of TNF-α in the cortex of the mammalian patient by administering to the patient an effective amount of a composition comprising pharmaceutically acceptable bodies of a size from about 20 nanometers (nm) to 500 micrometers (μm), comprising a plurality of phosphate-glycerol (PG) head groups or groups convertible to PG head groups.

3. The method according to claim 2, wherein said composition is essentially free of non-lipid pharmaceutically acceptable entities.

4. The method according to claim 2, wherein said PG head groups comprise from about 60 to 100% of head groups on said bodies.

5. The method according to claim 4, wherein said PG head groups comprise from about 75% of head groups on said bodies.

6. The method according to claim 4, wherein any remaining head groups comprise phosphate-choline groups.

7. The method according to claim 4, wherein the bodies are administered in a dosage amount comprising from about 500 to about 2.5×10⁹ bodies.

8. The method according to claim 7, wherein the bodies are liposomes comprising phosphatidylglycerol, with exteriorly presented phosphate-glycerol groups.

9. The method according to claim 8, wherein the liposomes have a size from about 20-1000 nm.

10. The method according to claim 9, wherein the liposomes comprise from about 60 to about 100% by weight phosphatidylglycerol, and correspondingly about 0 to about 40% by weight phosphatidylcholine.


SEQUENCE LISTING (4 sequences)

SEQ ID NO: 1 - DNA, 21 bp, Artificial Sequence
  Description of Artificial Sequence Synthetic Primer
cagcacttcttgtctttttgg

SEQ ID NO: 2 - DNA, 20 bp, Artificial Sequence
  Description of Artificial Sequence Synthetic Primer
ccaaggagctccagaaacag

SEQ ID NO: 3 - DNA, 21 bp, Artificial Sequence
  Description of Artificial Sequence Synthetic Primer
gtgggccgctctaggcaccaa

SEQ ID NO: 4 - DNA, 24 bp, Artificial Sequence
  Description of Artificial Sequence Synthetic Primer
ctctttgatgtcacgcacgatttc